(19) United States
(12) Patent Application Publication
(10) Pub. No.: US 2011/0034792 A1
(43) Pub. Date: Feb. 10, 2011

(54) Noninvasive Body Chemistry Monitor and Method

(76) Inventors: Ronald L. Williams, Fallbrook, CA (US); Robert Edwin Nasburg, Palos Verdes Estates, CA (US)

Correspondence Address: SoCAL IP LAW GROUP LLP, 310 N. WESTLAKE BLVD. STE 120, WESTLAKE VILLAGE, CA, 91362, US

(21) Appl. No.: 12/536,414

(22) Filed: Aug. 5, 2009

(51) Int. Cl. A61B 5/145

(52) U.S. Cl. 600/365; 600/309

(57) ABSTRACT

There is disclosed a noninvasive body chemistry monitor and method. A sensor may be disposed in close gaseous communication with the subject's skin. The sensor may be configured to provide a sensor output signal indicative of a level of at least one gaseous material released through the subject's skin. An event detector may detect a body chemistry event based on the sensor output signal.

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

1. Field

This disclosure relates to body chemistry monitors and, in particular, to noninvasive monitors to alert diabetics of abnormal blood glucose levels.

2. Description of the Related Art

Traditionally, blood glucose levels have been measured by analysis of a liquid blood sample. Commonly, blood glucose is measured by the so-called “stick” method. The subject's skin is pierced and a drop of blood is placed on a test strip. The test strip, with the blood sample, is introduced into a meter than performs an electrochemical process to determine blood glucose level.

Continuous blood glucose monitoring devices are also available. One such device employs a sensor including a thin electrode that is inserted under the subject's skin. While this approach still requires penetration of the skin, the sensor may be left in place to provide continuous blood glucose monitoring over an extended period of time such as a day or a week.

A variety of noninvasive blood glucose devices are being developed. One such device extracted a liquid analyte sample through the intact skin by means of reverse iontophotoresis, which is to say a liquid sample was extracted through the skin by application of a direct electric current. The liquid analyte was then subjected to electrochemical analysis to determine blood glucose level. While the skin was not pierced, the direct contact between the sensor and the skin and the use of an electric current is believed to have caused skin irritation and other adverse reactions in at least some subjects. Other published developmental techniques for noninvasive blood glucose monitoring include measuring the electrical conductance, the microwave resonance, or the optical reflectivity of the subject's skin.

### DESCRIPTION OF THE DRAWINGS

FIG. 1 is a schematic side view of a body chemistry monitor.

FIG. 2 is a block diagram of a body chemistry monitor.

FIG. 3 is a block diagram of a body chemistry monitor.

FIG. 4 is a graph showing test results for an experimental body chemistry monitor.

FIG. 5 is a flow chart of method for monitoring body chemistry.

Throughout this description, elements appearing in figures are assigned three-digit reference designators, where the most significant digit is the figure number and the two least significant digits are specific to the element. An element that is not described in conjunction with a figure may be presumed to have the same characteristics and function as a previously-described element having a reference designator with the same least significant digits.

### DETAILED DESCRIPTION

Description of Apparatus

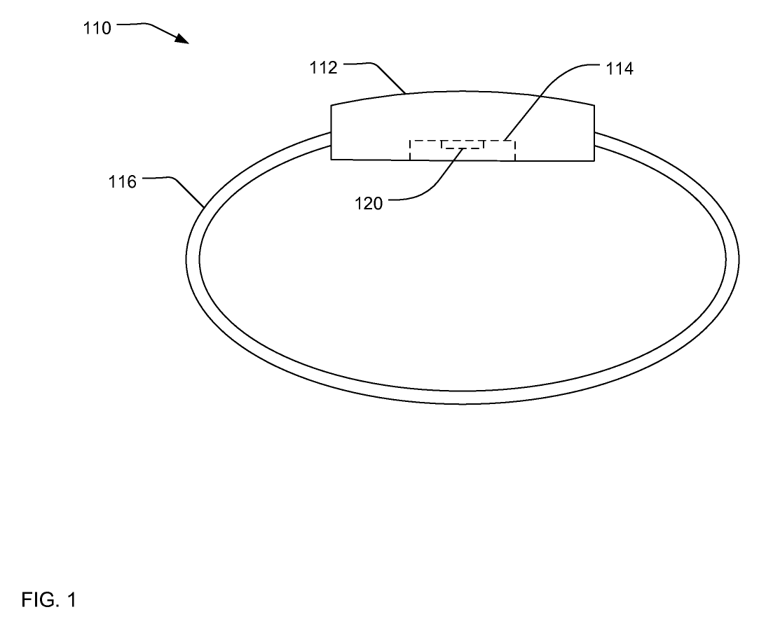

Referring now to FIG. 1, a noninvasive body chemistry monitor 110 may be adapted to measure at least one aspect of the body chemistry of a person, herein termed the “subject”. The noninvasive body chemistry monitor 110 may include a housing 112 which may enclose various elements of the body chemistry monitor. The housing 112 may be held against a subject's skin by a strap 116 or by another mechanism. The body chemistry monitor 110 may be in the general configuration of a wristwatch, a belt, or some other configuration which retains the housing 112 in contact with the subject's skin.

The noninvasive body chemistry monitor 110 may include at least one sensor 120 in close gaseous communication with the subject's skin. In this patent, the term “close gaseous communication” encompasses any configuration where the sensor is disposed proximate the user's skin and coupled to the skin by an open passage such that at least a portion of gaseous chemicals released through the subject's skin is highly likely to impinge upon the sensor. The term “released through the subject's skin” encompasses both gaseous chemicals that are exhausted directly through the skin and chemicals that are conducted through the skin in perspiration and then evaporate. The sensor 120 may be may be disposed adjacent to the subject's skin surface, or may be coupled to the subject's skin surface through an inlet or passage through which gaseous chemicals may flow. For example, the sensor 120 may be wholly or partially disposed in a cavity or recess 114 of the housing 110 proximate to the subject's skin.

The sensor 120 may be adapted to sense at least one gaseous chemical species that may be released through the subject's skin. The sensor 120 may be a device which experiences a change in electrical properties, such as resistance, conductivity, capacitance, resonance frequency, when exposed to one or more volatile chemical compounds. The sensor 120 may be a device that measures a property, such as optical transmission, of the gas evolved from the subject's skin directly. The sensor may be a metal oxide gas sensor, a conductive polymer gas sensor, an optical gas sensor, a spectrographic gas sensor, an electrochemical gas sensor, or other type of electronic nose. The body chemistry monitor 100 may include a plurality of sensors of the same or different types. The body chemistry monitor 100 may, for example, sense a specific chemical species by combining the outputs from two or more sensors.

Subjects with diabetes mellitus (diabetes) are susceptible to a potentially serious condition called ketoacidosis in which excessive levels of ketones, including acetone, build up in their blood. Ketoacidosis may occur when the body metabolizes fat, rather than carbohydrates, to produce energy. Mild ketoacidosis may occur naturally in subjects that do not have diabetes. For example, mild ketoacidosis may occur during strenuous exercise or in subjects on very low carbohydrate diets. According to the American Diabetes Association web site, a subject with diabetes may experience potentially harmful ketoacidosis due to insufficient insulin or due to excessive insulin in their body. Insufficient insulin may prevent normal metabolizing of glucose, resulting in high blood glucose levels. Excess insulin may result in very low blood glucose levels. In either circumstance, the body may be required to metabolize fat to produce energy, thus causing ketones to accumulate in the body.

When the noninvasive body chemistry monitor 110 is intended for monitoring blood glucose levels in a subject with diabetes, the sensor 120 may be configured to detect acetone and/or other ketones released through the subject's skin. The sensor 120 may be, for example, a metal oxide gas sensor, such as a so-called “Taguchi” gas sensor (TGS) available from Figaro Engineering Inc. of Osaka, Japan. Similar solid-state gas sensors may be available from International Sensor Technology of Irvine, Calif. The sensor 120 may be some other type of sensor. The sensor 120 may be specifically optimized for detection of acetone.

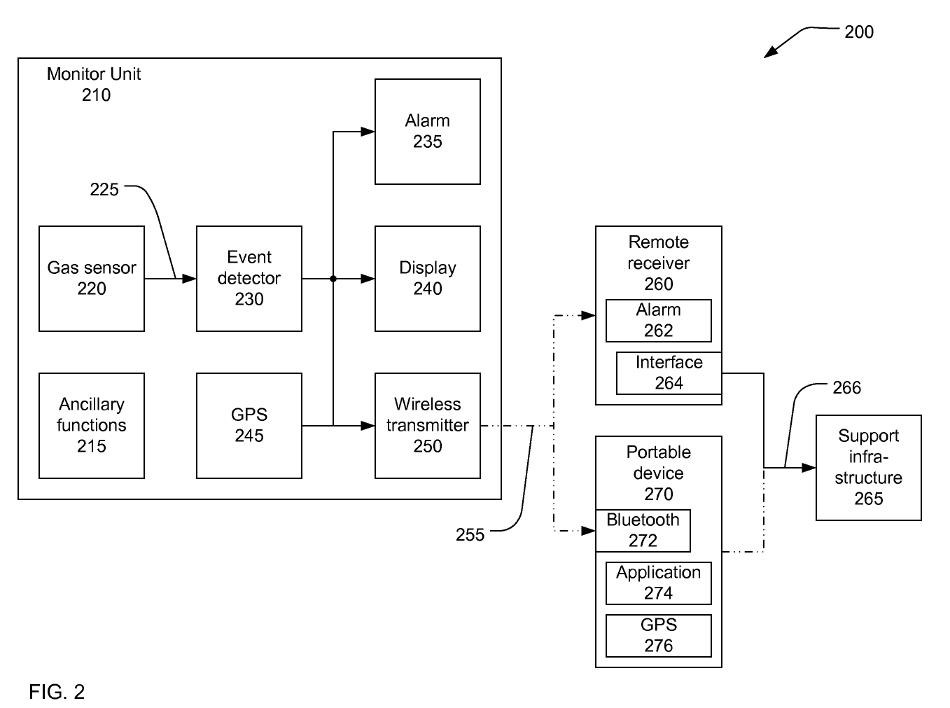

Referring now to FIG. 2, a noninvasive body chemistry monitoring system 200 may include a monitor unit 210, which may be the noninvasive body chemistry monitor 110. The monitor may be worn by a subject, which is to say that the monitor unit may be disposed such that a portion of the monitor unit is in contact with the subject's skin. The monitor unit 210 may include a gas sensor 220, which may be the sensor 120, and an event detector 230. The event detector 230 may be configured to detect a body chemistry event based on one or more signals received from the gas sensor 220. In this patent, the term “body chemistry event” means a detectable body chemistry condition that warrants generating an alarm or otherwise notifying a subject wearing the monitor unit. A body chemistry event may be, for example, a condition in which a concentration of one or more chemical species released through the subject's skin is too high, too low, or changing too quickly.

The event detector 230 may detect a body chemistry event by comparing at least one signal 225 received from the gas sensor 220 to one or more predetermined event criteria. The predetermined event criteria may include comparing a value, a time-integrated value, or a rate-of-change of the signal 225 to a fixed or adaptive threshold or range, and determining that a body chemistry event has occurred if the signal value exceeds the threshold or falls outside of the range. The event detector 230 may perform processing on the signal 225 to remove noise and/or spurious signals prior to comparing the signal 225 to the event criteria.

For example, a body chemistry event may be detected if the value of the signal 225 exceeds a first threshold, which may be fixed or adaptive. An adaptive threshold may be based on prior history and/or on some other parameter such as ambient temperature or the subject's body temperature. An adaptive threshold may be, for example, a multiple of an average value of the signal over a predetermined preceding time interval. Methods from target detection, pattern recognition, and other signal processing systems may be employed for adaptation. A body chemistry event may be detected if the rate of change of the signal 225 exceeds a second threshold, which also may be fixed or adaptive. For further example, a body chemistry event may be detected if the signal 225 changes, within a predetermined time interval, by more than a predetermined fraction of the average level of the signal 225.

The monitor unit 210 may include additional sensors not shown in FIG. 2. When the monitor unit includes multiple sensors, the event detector 230 may detect a body chemistry event based on signals received from the multiple gas sensors. For example, the event detector may detect a body chemistry event based on a difference between two sensor output signals received from two or more sensors or a ratio of sensor output signals received from two or more sensors.

The monitor unit 210 may include an alarm 235 which may be activated by the event detector 230 upon detection of a body chemistry event. The alarm 235 may cause, for example, an audible sound, a visible indication such as a blinking light, a physical effect such as vibration or electric shock, or a combination of these. The monitor unit may include a display 240 which may display, under control of the event detector 230, quantitative or qualitative information derived from the signal 225 and/or status information.

The monitor unit 210 may include a wireless transmitter 250 for transmitting messages to a remote receiver 260 and/or a portable device 270 via a communications channel 255. The wireless transmitter 250 may be, for example, a Bluetooth transmitter, a wireless personal area network transmitter in accordance with an IEEE 802.15.4 specification, a WiFi transmitter in accordance with an IEEE 802.11 specification, or some other form of wireless transmitter. The wireless transmitter 250 may be a wireless transceiver including a receiver. The wireless transmitter 250 may be, for further example, configured to place a call to a predetermined telephone number via a commercial wireless telephone network. The communications channel 255 may include a wired connection between the monitor unit 210 and the remote receiver 260 in addition to, or instead of, a wireless connection.

Upon detection of a body chemistry event, the event detector 230 may send at least one event message via the communication channel 255. In this context, the term “event message” encompasses any message sent by the monitor unit 210 when a body chemistry event is detected. The event message may include information identifying the subject wearing the monitor unit 210. The event message may include a serial number or other identification of the monitor unit. The event message may include information regarding the body chemistry event, such as a time of onset of the event, duration of the event, and/or quantitative information derived from the signal 225. In the case where the communication channel 255 is a commercial wireless telephone network, the wireless transmitter 260 may send the event message as, for example, a text message, an e-mail, or a prerecorded or synthesized voice message.

Upon detection of a body chemistry event, the event detector 230 may undertake a staged response. For example, the event detector may initially activate the alarm 235 and subsequently send an event message or a series of event messages via the communication channel 255 if the subject does not acknowledge the alarm or if the body chemistry event endures beyond a predetermined time limit.

The monitor unit 210 may also transmit one or more status messages via the communication channel 255 to inform the remote receiver 260 and/or portable device 270 that the monitor unit 210 is active and functional. A status message may be sent initially to register or log-in the monitor unit with the remote receiver. Subsequent status messages may be sent periodically to confirm the connection between the monitor unit 210 and the remote receiver. When the monitor unit 210 has reception capability, the monitor unit 210 may receive, in response to status messages, acknowledgement messages from the remote receiver 260 and/or the portable device 270.

The monitor unit 210 may include a GPS receiver 245 or other navigation device that can determine the location of the subject wearing the monitor unit 210. When a navigation device is present, the event message and/or the status messages may include information defining the location of the subject.

The event detector 230 may include software and/or hardware for providing functionality and features described herein. The event detector 230 may include one or more of: logic arrays, memories, analog circuits, digital circuits, software, firmware, and processors such as microprocessors, field programmable gate arrays (FPGAs), application specific integrated circuits (ASICs), programmable logic devices (PLDs) and programmable logic arrays (PLAs). The hardware and firmware components of the event detector 230 may include various specialized units, circuits, software and interfaces for providing the functionality and features described here. The processes, functionality and features may be embodied in whole or in part in software which operates on a processor. The hardware and software and their functions may be distributed such that some components are performed by a processor and others by other devices.

The monitor unit 210 may include additional software and/or hardware 215 for providing functions unrelated to or ancillary to body chemistry monitoring. For example, the monitor unit 210 may function as a watch, an exercise monitor, a music player, a telephone, or a navigation system concurrently with performing the functions of a body chemistry monitor.

The noninvasive body chemistry monitoring system 200 may include a remote receiver 260 configured to receive event and status messages from the monitor unit 210 via the communications channel 255. The remote receiver may be dedicated to the body chemistry monitoring system 200, and may be located, for example, in the subject's home, office, or automobile. The body chemistry monitoring system 200 may include a plurality of remote receivers (not shown in FIG. 2) disposed in locations frequented by the subject. A remote receiver may be a portion of a home security system or a medical alert system.

The remote receiver 260 may include an alarm 262 which may be activated upon receipt of an event message from the monitor unit 210 indicating detection of a body chemistry event. The remote unit 260 may include an interface 264 coupled to a second communications channel 266 to send a relayed event message relating to the body chemistry event to a support infrastructure 265. The relayed event message may relay, or repeat, all or portions of the event message received from the monitor unit 210. The relayed event message may include, for example, the identification of the subject wearing the monitor unit 210, a notification that a body chemistry event has occurred, qualitative or quantitative information relating to the severity or duration of the body chemistry event, and information indicating the location of the subject if available. The support infrastructure may, for example, include the subject's family or friends, the subject's physician, emergency medical services personnel, a service dedicated to supporting a plurality of body chemistry monitoring systems, or some other infrastructure organization that can provide support to the subject during a body chemistry event.

The remote receiver 260 may also send remote receiver status (RRS) messages to the support infrastructure 265 via the second communications channel. The RRS messages may indicate that the remote receiver is active and functional, and may also indicate that the remote receiver is receiving status messages from the monitor unit 210. When the remote receiver 260 fails to receive one or more status messages from the monitor unit 210, the remote receiver 260 may send an RRS message to the support infrastructure indicting the subject is no longer being monitored.

The second communications channel 266 may be or include a network, which may be the Internet, a local area network, a wide area network, or some other network. The second communications channel 266 may be or include a wired or wireless telephone system. The remote receiver 260 may transmit the relayed event message to the support infrastructure 265 by sending a text message, by sending an e-mail, or by accessing a web site. When the second communications channel 266 is a wired or wireless telephone system, the relayed event message may be an e-mail, text message, and/or a synthesized or prerecorded message sent via a conventional telephone call to a predetermined telephone number.

The body chemistry monitoring system 200 may include a portable device 270, which may be a cell phone, a smart phone, an e-mail appliance, a portable computer, or other device capable of wireless communications with a network such as a cell phone network or the Internet. The portable device 270 may be included in the body chemistry monitoring system 200 in addition to, or instead of, one or more remote receiver 260. In addition to wireless communications with the network, the portable device 270 may be adapted to communicate wirelessly with the monitor unit 210. For example, the portable device 270 may include a Bluetooth transceiver 272 to receive event and status messages from the monitor unit 210 via the communications channel 255.

The portable device 270 may include an application 274, stored in a memory or storage device. When executed, the application 274 may cause the portable device 270 to monitor the Bluetooth transceiver to determine if an event message has been received from the monitor unit 210 and, when an event message is received, to take responsive action. The responsive action may include generating an alarm, for example by means of a distinctive ring tone. The responsive action may further include sending a relayed event message to the support infrastructure via the second communications channel 266. The responsive action may include sending a plurality of relayed event messages to a corresponding plurality of destinations via the network.

The portable device 270 may also monitor status messages transmitted by the monitor unit 210. The portable device may take responsive action, such as sending a message to the support infrastructure 265 indicating that the subject is not being monitored, if anticipated status messages are not received.

The portable device 270 may include a GPS receiver or other navigation device that provides location information indicting the location of the portable device. When a navigation device is present, the relayed event messages and/or RSS messages sent by the portable device may include the location information.

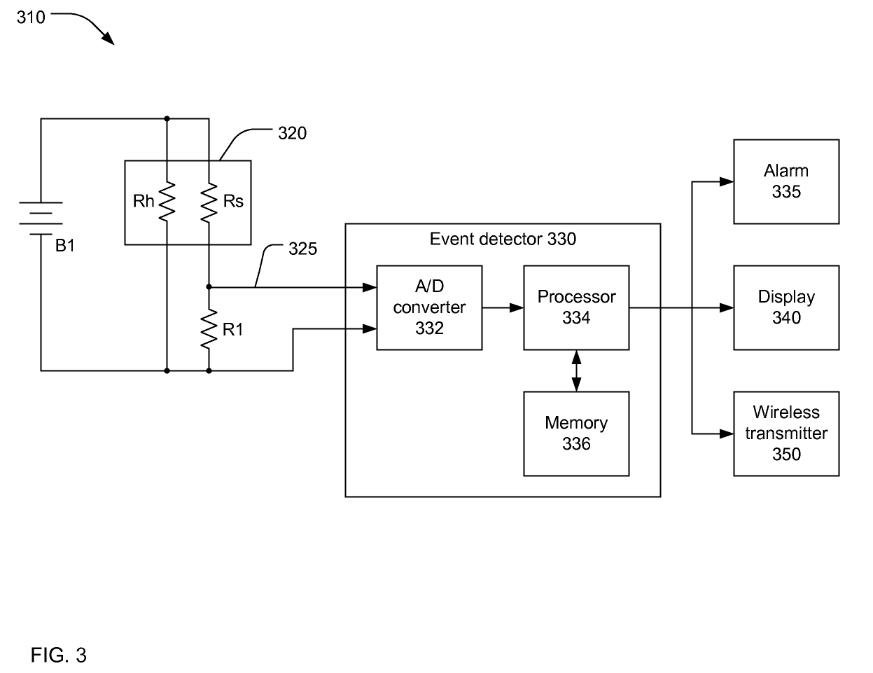

An exemplary monitor unit 310, which may be suitable for the monitor unit 210, may include one or more metal oxide gas sensor 320 adapted to sense acetone and/or other ketones released through a subject's skin. The metal oxide gas sensor 320 may include a heater Rh and a sensing resistor Rs. The heater Rh may be connected to an appropriate power source, represented in FIG. 3 by the battery B1, to maintain the temperature of the metal oxide gas sensor 320 at a predetermined temperature. The predetermined temperature may be a temperature determined to be optimal for sensing acetone and/or other ketones. The resistance value of the sensing resistor Rs may change depending on the concentration of acetone and/or ketones in the gas proximate to the sensing element.

The sensing resistor Rs may be connected in series with a resistor RI to form a resistive voltage divider connected in parallel with the battery B1 or another voltage source. The resistance value of the sensing resistor Rs, and thus the voltage 325 at the junction of Rs and R1, may change depending on the concentration of acetone and/or ketones in the gas proximate to the sensing element.

An event detector 330 may determine if a body chemistry event has occurred based on the voltage 325. In the example of FIG. 3, the event detector 330 may include a analog to digital (A/D) converter 332 to transform the analog voltage 325 into digital data representative of the voltage 325. A processor 334 may determine if a body chemistry event has occurred based on the digital data from the A/D converter 332. The processor 334 may be coupled to a memory 336. The processor 332 may execute a program stored in a memory 336, and may also store data in the memory 336. The program executed by the processor may include filtering the digital data from the A/D converter to exclude or reduce noise and spurious values, integrating the digital data to form a time-averaged value, setting one or more adaptive thresholds based on the time averaged value and/or other parameters such as ambient temperature or the subject's skin temperature, determining a rate of change of the digital data, and comparing the digital data and/or the rate of change of the digital data to one or more fixed or adaptive threshold. Upon determination that a body chemistry event has occurred, event detector may execute additional program instructions to activate an alarm 335, to present information relating to the body chemistry event on a display 340, and/or to send an event message via a wireless transmitter 350.

The processor 334 may perform other functions including controlling the transmission of periodic status messages via the wireless transmitter 350, and power management. The processor 334 may manage the power consumed by the monitor unit 310, in part, by controlling a sampling rate at which the output of the sensor 320 is sampled and processed.

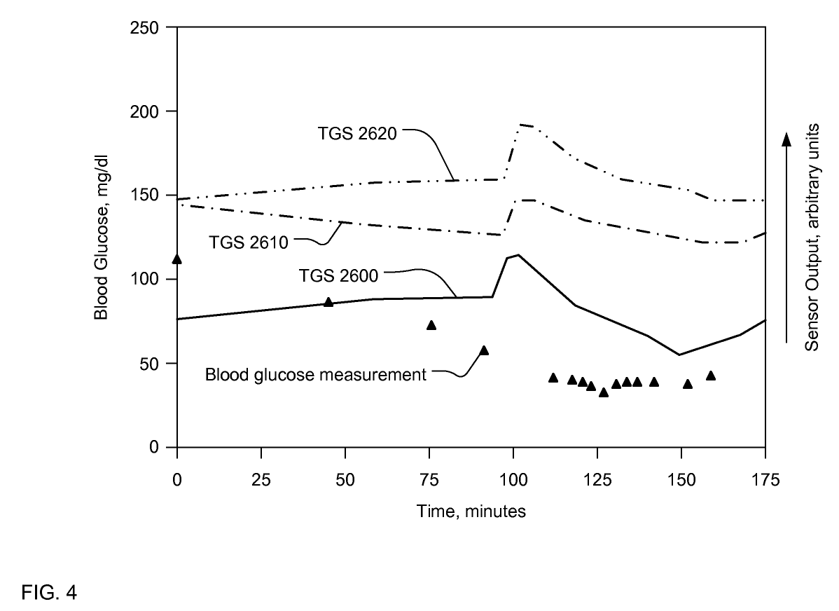

Turing now to FIG. 4, results from an experimental body chemistry monitor unit demonstrate the possibility of sensing an insufficient blood glucose event using a solid state gas sensor. The experimental unit included commercially-available TGS 2600, TGS 2610, and TGS 2620 metal oxide gas sensors available from Figaro Engineering Inc. of Osaka, Japan. The three gas sensors were disposed in an enclosure having an opening placed in contact with the subject's skin. Each sensor included a heater and a sensing resistor connected as shown in FIG. 3. Each sensing resistor was connected in series with another resistor to form a voltage divider. The output of each sensor, corresponding to the voltage 325 in FIG. 3, was measured with a voltage measuring device and is plotted as a function of time in FIG. 4. Also plotted in FIG. 4 are periodic blood glucose measurements made using the “stick” method.

At time t=0, the subject received an injection of human analog insulin sufficient to drive the subject's blood glucose to an abnormally low level. The outputs from all three sensors exhibited a rapid increase at time about 100 minutes, roughly coincident with the subject's blood glucose level dropping to less than 60 mg/dl. When blood glucose level are sufficiently low, the subject's body may transition into an emergency state in which the body's chemistry may change rapidly to raise the blood glucose level back to normal. The rapid change in the sensor outputs at time≈100 minutes may be due to a surge in acetone levels in the subject due to metabolism of fat and/or other chemical changes indicative of a low blood glucose body chemistry event. Shortly after this time, the subject began ingesting sugar-containing food to recover to a normal blood sugar level.

The output from the TGS 2600 sensor exhibited a rapid increase about one minute before the outputs from the other two sensors. The output from the TGS 2610 sensor apparently tracked the blood glucose level from the start of the test at time zero to the rapid increase indicative of the body chemistry event. The performance of the TGS 2610 sensor may indicate that an optimized gas sensor may provide quantitative measurements of blood glucose levels as well as a qualitative detection of body chemistry events.

Description of Processes

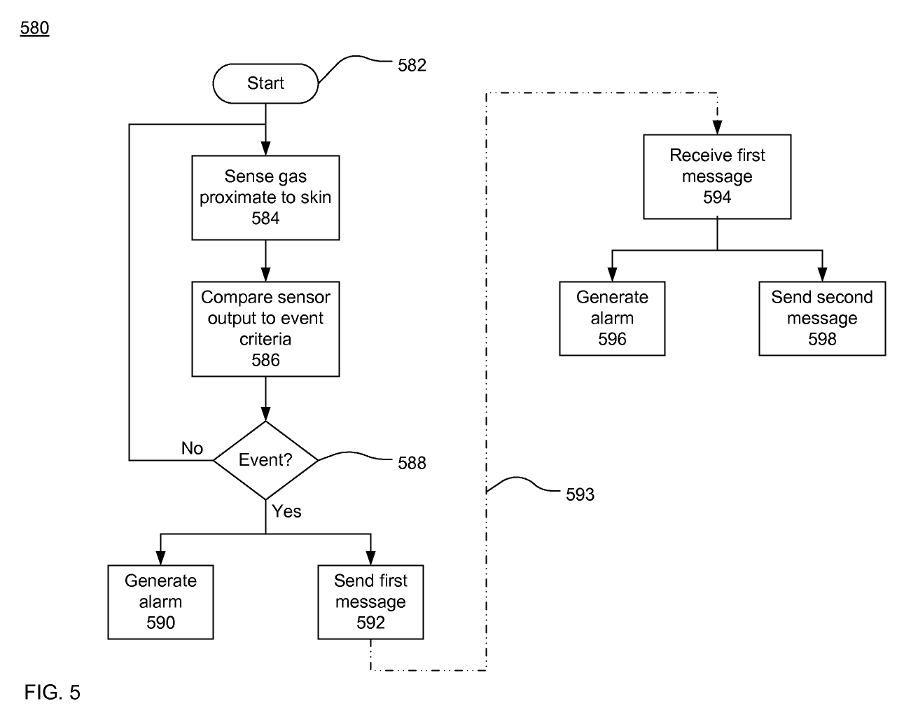

Referring now to FIG. 5, a flow chart of a process 580 for detecting body chemistry events may start at 582, for example when a subject puts on a wearable monitor unit, and may proceed continuously or until such time (not shown in FIG. 5) as the subject removes the monitor until or the monitor unit otherwise ceases to operate.

At 584, at least one sensor disposed in close gaseous communication with the subject's skin may sense at least one gaseous chemical released through the subject's skin. The sensor may provide a sensor output signal indicative of, for example, a concentration of the sensed gaseous chemical proximate the subject's skin.

At 586, at least one sensor output signal may be compared to one or more predetermined event criteria. Processing may be performed on the sensor output signal to remove noise and/or spurious signal components prior to comparing the sensor output signal to the predetermined event criteria. The predetermined event criteria may include, for example, one or more of a maximum value, a minimum value, a maximum time-integrated value, a minimum time-integrated value, and a maximum rate-of-change of the sensor output signal. The predetermined event criteria may include, for further example, one or more of a maximum value, a minimum value, a maximum time-integrated value, a minimum time-integrated value, and a maximum rate-of-change of the difference, ratio, or other combination of sensor output signals from two or more sensors. The various maximum and/or minimum values may be fixed or adaptive. An adaptive maximum or minimum value may be based on prior history and/or on some other parameter such as ambient temperature or the subject's body temperature. An adaptive maximum may be, for example, a multiple of an average value of the signal over a predetermined preceding time interval.

At 588, a determination may be made if a body chemistry event has been detected. When an event has not been detected, the process may return to 584 and repeat continuously. Although the actions at 584, 586, and 588 are shown as sequential for ease of description, these actions may performed essentially simultaneously.

When a determination is made at 588 that a body chemistry event has been detected, an alarm may be generated at 590 and/or an event message, as previously described, may be sent wirelessly at 592. An alarm may be generated at 590 and an event message may be sent at 592 concurrently. An alarm may be generated at 590 upon determination that a body chemistry event has been detected, and en event message may be subsequently sent at 592 if the alarm is not acknowledged within a predetermined time interval, or if the body chemistry event persists beyond a predetermined duration.

The event message may be received by a remote device, which may be a portable device, at 594. The remoter device may generate an alarm at 596. The remote device may send relayed event message, as previously described, at 598.

Closing Comments

Throughout this description, the embodiments and examples shown should be considered as exemplars, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items.

It is claimed:

1. A noninvasive body chemistry monitor, comprising:
a sensor disposed in close gaseous communication with a subject's skin, the sensor configured to provide a sensor output signal indicative of a level of at least one gaseous material released through the subject's skin
an event detector to detect a body chemistry event based on the sensor output signal.

2. The body chemistry monitor of claim 1, wherein the sensor is selected from the group consisting of a metal oxide gas sensor, a conductive polymer gas sensor, an optical gas sensor, a spectrographic gas sensor, an electrochemical gas sensor, and an electronic nose.

3. The body chemistry monitor of claim 2, wherein the sensor is a metal oxide gas sensor configured to detect acetone the body chemistry event is at least one of a high blood glucose level and a low blood glucose level.

4. The body chemistry monitor of claim 1, further comprising:
an alarm coupled to the event detector, wherein the event detector is configured to activate the alarm when a body chemistry event is detected.

5. The body chemistry monitor of claim 1, further comprising:
a wireless transmitter coupled to the event detector, wherein the event detector sends an event message via the wireless transmitter when a body chemistry event is detected.

6. The body chemistry monitor of claim 5, wherein
the wireless transmitter sends the event message to a predetermined destination via a network.

7. The body chemistry monitor of claim 5, wherein
the wireless transmitter sends the event message to a remote unit configured to perform, upon receipt of the message, at least one of activating an alarm and sending a relayed event message to a predetermined destination via a network.

8. The body chemistry monitor of claim 7, wherein
at least one of the body chemistry monitor and the remote unit includes a GPS receiver to provide the location of the subject.

9. The body chemistry monitor of claim 7, wherein
the remote unit is a portable device configured for wireless communications via a network
the wireless transmitter sends the event message to the portable device via a Bluetooth connection.

10. The body chemistry monitor of claim 9, wherein the portable device contains an application which, upon receipt of the message, causes the portable device to send a relayed event message to a predetermined destination via the network.

11. The body chemistry monitor of claim 1, wherein the event detector detects a body chemistry event based on one or more of the level of the sensor output signal and the rate of change of the sensor output signal.

12. The body chemistry monitor of claim 1, wherein
the sensor is a plurality of sensors
the event detector detects a body chemistry event based on a corresponding plurality of sensor output signals from the plurality of sensors.

13. The body chemistry monitor of claim 12, wherein the event detector detects a body chemistry event based a difference or a ratio of two sensor output signals from two sensors.

14. The body chemistry monitor of claim 1, wherein the sensor and the event detector are disposed within a housing adapted to be worn in contract with a subject's skin.

15. The body chemistry monitor of claim 14, wherein the housing is configured generally in the form of a wristwatch.

16. A method for monitoring body chemistry, comprising:
disposing a sensor in close gaseous communication with a subject's skin, the sensor configured to provide a sensor output signal indicative of a level of at least one gaseous material released through the subject's skin
detecting a body chemistry event based on the sensor output signal.

17. The method for monitoring body chemistry of claim 16, wherein the sensor is selected from the group consisting of a metal oxide gas sensor, a conductive polymer gas sensor, an optical gas sensor, a spectrographic gas sensor, an electrochemical gas sensor, and an electronic nose.

18. The method for monitoring body chemistry of claim 17, wherein
the sensor is a metal oxide gas sensor configured to detect acetone
the body chemistry event is at least one of a high blood glucose level and a low blood glucose level.

19. The method for monitoring body chemistry of claim 16, further comprising:
activating an alarm to notify the subject when a body chemistry event has been detected.

20. The method for monitoring body chemistry of claim 16, further comprising:
sending a wireless event message when a body chemistry event has been detected.

21. The method for monitoring body chemistry of claim 20, wherein
the event message is transmitted to a predetermined destination via a cell phone network.

22. The method for monitoring body chemistry of claim 20, wherein
the event message is a message to a remote unit
upon receipt of the event message, the remote unit performing one or more of activating an alarm and sending a relayed event message to a predetermined destination via a network.

23. The method for monitoring body chemistry of claim 22, wherein
the remote unit includes a GPS receiver
upon receipt of the event message, the remote unit sending the relayed event message including the location of the subject to a predetermined destination via a network.

24. The method for monitoring body chemistry of claim 23, wherein
the remote unit is a portable device configured for wireless communications with the network
the wireless transmitter sends the event message to the portable device via a Bluetooth connection.

25. The method for monitoring body chemistry of claim 16, wherein detecting a body chemistry event based on the sensor output signal further comprises:
detecting the body chemistry event based on one or more of the level of the sensor output signal and the rate of change of the sensor output signal.

26. The method for monitoring body chemistry of claim 16, wherein
the sensor is a plurality of sensors
detecting a body chemistry event further comprises detecting the body chemistry event based on a difference or a ratio of two sensor output signals from two sensors.
